(19) United States
(12) Patent Application Publication
(10) Pub. No.: US 2024/0417644 A1
(43) Pub. Date: Dec. 19, 2024

(54) PHENOLAMINE ADDITIVE, PREPARATION METHOD AND USE THEREOF

(71) Applicant: Francool Technology (Shenzhen) Co., Ltd., Shenzhen (CN)

(72) Inventors: Yang Zhao, Shenzhen (CN); Chao Zhao, Shenzhen (CN); Wei Zhang, Shenzhen (CN); Jun Zhang, Shenzhen (CN); Dexiu Liu, Shenzhen (CN)

(21) Appl. No.: 18/822,336

(22) Filed: Sep. 2, 2024

(30) Foreign Application Priority Data
Aug. 13, 2024 (CN) 202411107998.5

(51) Int. Cl. C10M 133/08; C07C 209/60; C10N 30/16

(52) U.S. Cl. CPC C10M 133/08; C07C 209/60; C10N 2030/16

(57) ABSTRACT

The present application falls within the technical field of additives for metalworking fluids and specifically relates to a phenolamine additive, a preparation method, and use thereof. The phenolamine additive comprises a compound of Formula (1), a compound of Formula (2), a compound of Formula (3), a compound of Formula (4), and a compound of Formula (5), which has the characteristics of good antimicrobial performance and high stability, and does not produce any sedimentation and precipitation when compounded into a metalworking fluid.

### CROSS-REFERENCE TO RELATED APPLICATIONS

This application claims priority to Chinese Patent Application No. 202411107998.5, filed on Aug. 13, 2024, the content of which is incorporated herein by reference in its entirety.

### TECHNICAL FIELD

The present application falls within the technical field of additives for metalworking fluids and specifically relates to a phenolamine additive, a preparation method, and use thereof.

### BACKGROUND

Metalworking fluids have the functions of cooling, lubrication, washing, rust prevention, etc., and also have good stability and can be stored for a long time without aggregation and sedimentation, which play an important role in workpiece working.

In practical production use, it is essential to maintain the stability of metalworking fluids. One of the most common problems with metalworking fluids is the decrease in pH, which can lead to instability of the processing fluid system and precipitation of active ingredients. The reason for the decrease in pH of the metalworking fluid is mainly the consumption of bacteria and the neutralization of carbon dioxide. The addition of microbicides to formulations to reduce bacterial consumption is a common approach taken by the industry.

The main functions of microbicides in metalworking fluids include the following:
1. Prevention of microbial contamination and growth: metalworking fluids are susceptible to contamination by microorganisms such as bacteria, fungi, and yeast during use. These microorganisms can rapidly proliferate in the working fluid, resulting in liquid deterioration, odorous, discoloration, and other problems. The microbicide can effectively inhibit or kill these microorganisms, and keep the working fluid clean and stable.
2. Improvement of workability and quality: the stable working fluid can maintain good cooling, lubrication, washing, and rust prevention properties to ensure the working quality of workpieces and tools, and avoid performance degradation caused by microbial contamination, which can affect working accuracy and surface quality.
3. Maintainment of hygienic working environment: metalworking fluids contaminated by microorganisms can emit bad odors, affecting the air quality of the working environment and the health of the operators. Microbicides can prevent deterioration of working fluids and maintain hygiene and comfort of the working environment.
4. Environmental requirements: with the increasingly stringent environmental regulations, manufacturers need to reduce the production and emissions of waste. Extending the service life of metalworking fluids reduces the generation of waste fluids, reduces the burden on the environment, and meets environmental requirements.

In conclusion, the application of microbicides in metalworking fluids is crucial, not only does it help to maintain the stability and performance of the fluids, but also improves productivity and quality, reduces costs, and safeguards operator health and environmental safety.

There are many kinds of microbicides in metalworking fluids. The microbicides required by different working environments should be determined according to their characteristics. However, the commonly used microbicides all have the disadvantage of lacking stability.

### SUMMARY

The technical problem to be solved by the present application is to provide a phenolamine additive with good antimicrobial performance and high stability, a preparation method, and use thereof.

Traditional microbicides are widely used, but they all have some limitations. Isothiazolinones (such as methyl isothiazolinone and methyl chloroisothiazolinone) are known for their broad-spectrum antimicrobial properties and low concentration effectiveness. For example, Chinese invention patent with application number of 202180075767.9 discloses an antiseptic composition for metalworking fluids, wherein 1,2-benzisothiazolin-3-one, and polyether triamine are used synergistically to improve the microbicidal activity of the isothiazolinone. However, isothiazolinone microbicides are irritating to the skin and have poor stability at high pH. Formaldehyde releasers, such as hexachloromethylene tetrahydrobenzodithiazole, provide long-lasting microbicidal effects at low cost, but formaldehyde release is harmful to health and odorous. Phenolic compounds (e.g. p-tert-butylphenol) and guanidine compounds (e.g. polyhexamethylene biguanide hydrochloride) have broad-spectrum antimicrobial properties and are stable over a wide pH range, but some phenols may be toxic and guanidine compounds are costly. Nitro compounds, such as Brobol, provide fast and effective sterilization but are unstable at high temperatures and may be toxic. Quaternary ammonium salts, such as benzalkonium chloride, are effective and safe in killing microorganisms at low concentrations but are less effective in the presence of organics and long-term use may lead to resistance. Silver and copper ions, while long-lasting, are costly and can corrode materials. It is necessary to comprehensively consider the characteristics and application environment to select suitable microbicides.

In order to solve the above-mentioned technical problem, the technical solution adopted by the present application is a phenolamine additive including a compound of Formula (1), a compound of Formula (2), a compound of Formula (3), a compound of Formula (4), and a compound of Formula (5);

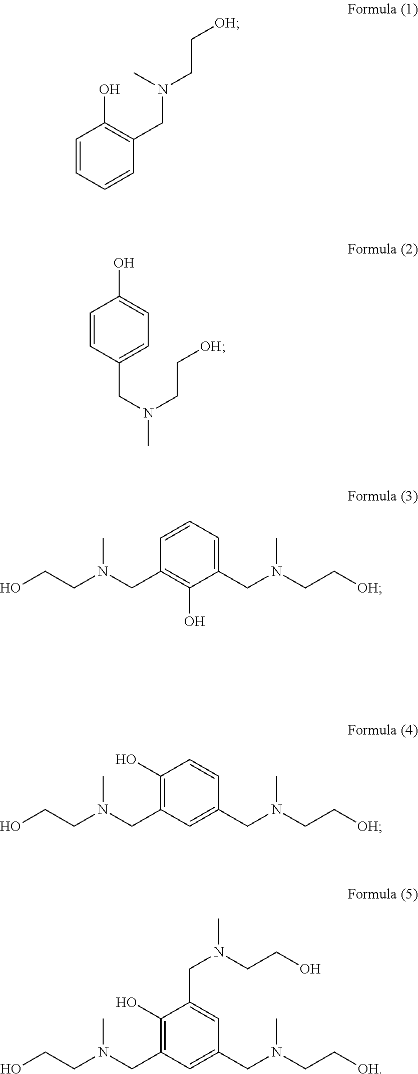 [SMILES: CN(CCO)Cc1ccccc1O ; CN(CCO)Cc1ccc(O)cc1 ; CN(CCO)Cc1cccc(CN(C)CCO)c1O ; CN(CCO)Cc1ccc(O)c(CN(C)CCO)c1 ; CN(CCO)Cc1cc(CN(C)CCO)c(O)c(CN(C)CCO)c1]

Another technical solution adopted by the present application is a preparation method of the above-mentioned phenolamine additive, comprising the steps of reacting phenol, methyl monoethanolamine, and formaldehyde by the Mannich reaction to obtain a compound of Formula (1), a compound of Formula (2), a compound of Formula (3), a compound of Formula (4), and a compound of Formula (5).

Another technical solution adopted by the present application is the use of the phenolamine additive described above as a microbicide in a metalworking fluid.

The beneficial effects of the present application include: the phenolamine additive of the present application comprising the compound of Formula (1), the compound of Formula (2), the compound of Formula (3), the compound of Formula (4), and the compound of Formula (5), has the characteristics of good antimicrobial performance and high stability, and does not produce any sedimentation and precipitation when compounded into a metalworking fluid.

### DETAILED DESCRIPTION OF THE EMBODIMENTS

In order to provide a detailed explanation of the technical content, achieved objectives, and effects of the present application, the following will be explained in conjunction with the implementation method.

A phenolamine additive comprises a compound of Formula (1), a compound of Formula (2), a compound of Formula (3), a compound of Formula (4), and a compound of Formula (5);

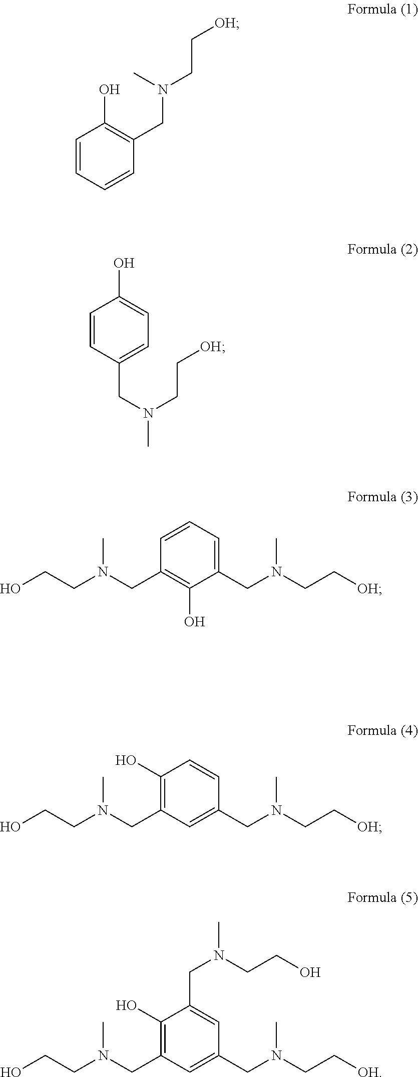 [SMILES: CN(CCO)Cc1ccccc1O ; CN(CCO)Cc1ccc(O)cc1 ; CN(CCO)Cc1cccc(CN(C)CCO)c1O ; CN(CCO)Cc1ccc(O)c(CN(C)CCO)c1 ; CN(CCO)Cc1cc(CN(C)CCO)c(O)c(CN(C)CCO)c1]

It can be seen from the above description that the beneficial effects of the present application are as follows: the structure of the phenolamine additive of the present application including two monoalcohol amines, two diol amines, and one trialcohol amine, which has good antimicrobial performance and a certain pH-adjusting ability, high stability, can be intermiscible with water in any proportion, and will not produce any sedimentation and precipitation when compounded into metalworking fluids. In addition, the phenolamine additives of the present application are all single-molecular species and have a better performance in terms of solubility.

Another technical solution adopted by the present application is a preparation method of the above-mentioned phenolamine additive, comprising the steps of reacting phenol, methyl monoethanolamine, and formaldehyde by the Mannich reaction to obtain a compound of Formula (1), a compound of Formula (2), a compound of Formula (3), a compound of Formula (4), and a compound of Formula (5).

It can be seen from the above-mentioned description that in the present application, phenol, methyl monoethanolamine, and formaldehyde are selected to prepare the phenolamine additive by the Mannich reaction. Phenol is selected because it has three reaction sites, high in reactivity, and large adjustable range in product ratio. Higher alkali values can be obtained by adjusting the reaction ratio, without the need to use a strong alkali. The obtained product is more compatible with the actual application of metalworking fluids with higher pH. Methyl monoethanolamine was selected as a reactant because methyl monoethanolamine has only one reaction site with phenol to form single-molecular species rather than a polymer and the product is more soluble.

The preparation method of the present application is low in cost, and the involved raw materials of phenol, formaldehyde, and methyl monoethanolamine all have a great price advantage in the market. The process is simple, the scheme is based on the Mannich reaction. The reaction condition is only heating, without the use of catalysts, special solvents, or special processes. The reaction is thorough and pollution-free.

Further, the following steps are included: water, methyl monoethanolamine, phenol, and formaldehyde are sequentially added to the reaction vessel and reacted at 85-95° C. for 3.8 to 4.2 h to obtain the compound of Formula (1), the compound of Formula (2), the compound of Formula (3), the compound of Formula (4), and the compound of Formula (5).

It can be seen from the above description that the reaction temperature is too low for the reaction, resulting in slow reaction speed or incomplete reaction, formaldehyde residue; too high temperature will lead to overheating of the system, leading to seriously yellowing the system. In addition, a large amount of water vapor will evaporate, resulting in inaccurate concentration. At the same time, phenol or formaldehyde vapors escapes, which is not conducive to the environment and human health.

Further, the mass of water is 15% of the total mass of methyl monoethanolamine, phenol, and formaldehyde.

Further, formaldehyde is added to the reaction vessel in batches.

It can be seen from the above description that the reaction of the present application is easy to occur, and adding formaldehyde too quickly would lead to a rapid temperature rise of the system, with the risk of overheating and boiling. Therefore, formaldehyde needs to be divided into batches in order to control the reaction rate.

Further, the mass ratio of methyl monoethanolamine to phenol is 1 to 2.5:1.

It can be seen from the above description that the distribution ratio of different substitution products is affected by the change in the ratio of methyl monoethanolamine to phenol. If the molar ratio of methyl monoethanolamine is less than phenol, there will be residual phenol in the system, causing the product to have an unpleasant odor.

Further, the mass ratio of formaldehyde to phenol is 0.4 to 0.9:1.

As can be seen from the above description, controlling the molar amount of formaldehyde to be greater than phenol and less than methyl monoethanolamine allows the reaction to be complete without residual formaldehyde or phenol.

Another technical solution adopted by the present application is the use of the phenolamine additive described above as a microbicide in a metalworking fluid.

It can be seen from the above-mentioned description that the phenolamine additive of the present application has excellent microbicidal performance and good stability when compounded into a metalworking fluid, without causing any sedimentation or precipitation problems.

In some examples: a phenolamine additive consists of a compound of Formula (1), a compound of Formula (2), a compound of Formula (3), a compound of Formula (4), and a compound of Formula (5);

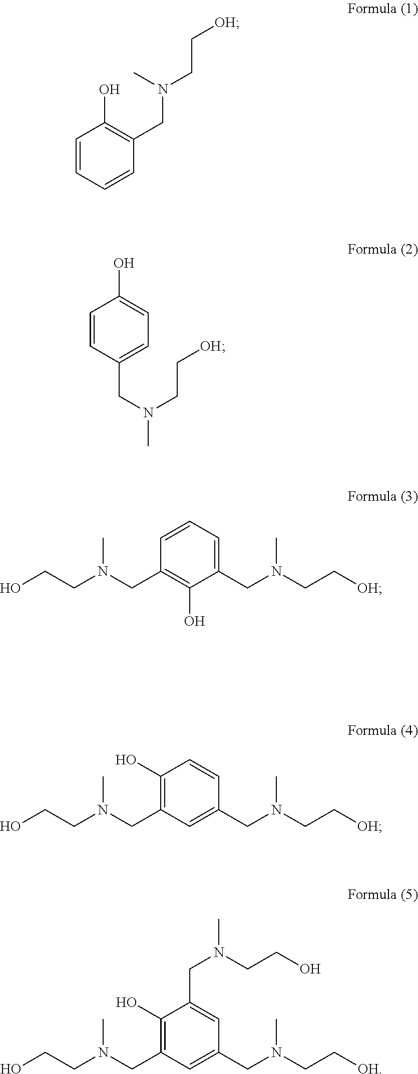 [SMILES: CN(CCO)Cc1ccccc1O ; CN(CCO)Cc1ccc(O)cc1 ; CN(CCO)Cc1cccc(CN(C)CCO)c1O ; CN(CCO)Cc1ccc(O)c(CN(C)CCO)c1 ; CN(CCO)Cc1cc(CN(C)CCO)c(O)c(CN(C)CCO)c1]

In some examples, the phenolamine additive of the above embodiments can be used as a microbicide in a metalworking fluid.

In some examples, a preparation method of a phenolamine additive included the following steps. 15.2 g of methyl monoethanolamine was added to a reaction vessel containing 5.4 g of water. 9.4 g of phenol was added to the reaction vessel and mixed to obtain a mixture. Then, 6 g of formaldehyde was added to the mixture in three portions and reacted at 90° C. for 4 h to obtain phenolamine additive 1. The reaction process is shown in the following chemical equation.

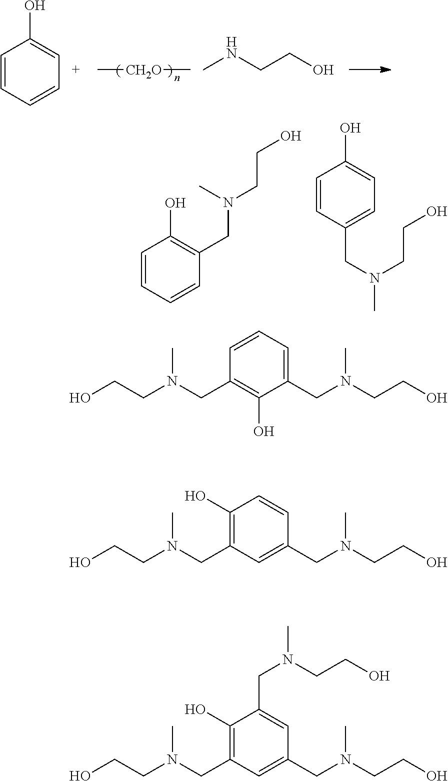

In some examples, a preparation method of a phenolamine additive included the following steps. 10 g of methyl monoethanolamine was added to a reaction vessel containing 6.2 g of water. 9.4 g of phenol was added to the reaction vessel and mixed to obtain a mixture. Then, 4 g of formaldehyde was added to the mixture in three portions and reacted at 85° C. for 4 h to obtain the phenolamine additive.

In some examples: a preparation method of a phenolamine additive included the following steps. 20 g of methyl monoethanolamine was added to a reaction vessel containing 3.1 g of water. 9.4 g of phenol was added to the reaction vessel and mixed to obtain a mixture. Then, 6 g of formaldehyde was added to the mixture in three portions and reacted at 95° C. for 4 h to obtain the phenolamine additive.

Testing: antimicrobial properties and stability tests were performed on the phenolamine additive 1 obtained from the preparation of the present application. A semi-synthetic metalworking fluid in the following mass percentages was used for the testing:

| Comp. Ex. | | Blank Ex. | | Test Ex. | |
|---|---|---|---|---|---|
| | Mass | | Mass | | Mass |
| Component | percentage | Component | percentage | Component | percentage |
| Phosphate corrosion | 2.5% | Phosphate corrosion | 2.5% | Phosphate corrosion | 2.5% |
| inhibitor | | inhibitor | | inhibitor | |
| Triethanolamine | 6% | Triethanolamine | 6% | Phenolamine additive 1 | 8% |
| Monoethanolamine | 3% | Monoethanolamine | 3% | Monoethanolamine | 3% |
| Isooctyl palmitate | 35% | Isooctyl palmitate | 35% | Isooctyl palmitate | 35% |
| Ricinoleic acid | 3% | Ricinoleic acid | 3% | Ricinoleic acid | 3% |
| Tall oleic acid | 2% | Tall oleic acid | 2% | Tall oleic acid | 2% |
| Dodecanedioic acid | 1.5% | Dodecanedioic acid | 1.5% | Dodecanedioic acid | 1.5% |
| Caprolactam | 1.5% | Caprolactam | 1.5% | Caprolactam | 1.5% |
| Benzisothiazolinone | 0.8% | Benzisothiazolinone | 0% | Benzisothiazolinone | 0% |
| Castor oil | 7% | Castor oil | 7% | Castor oil | 7% |
| Glyceryl oleate | 7% | Glyceryl oleate | 7% | Glyceryl oleate | 7% |
| Polyether | 8% | Polyether | 8% | Polyether | 8% |
| Defoamer | 2% | Defoamer | 2% | Defoamer | 2% |
| Water | 20.7% | Water | 21.5% | Water | 19.5% |

Description: the additive in the present application has both pH adjustment and microbicidal functions. Compared with the traditional semi-synthetic formula of metalworking fluid (comparative example), the test example did not add the traditional benzisothiazolinone microbicide and triethanolamine, and the addition amount of phenolamine additive 1 was 8% in order to maintain the same alkali value; the blank example does not add a microbicide (benzisothiazolinone), and the rest was the same as the comparative example; the water content of the comparative example, the blank example and the test example was made up to 100%.

Test Result 1: basic data comparison (within 48 h). The comparison results are as follows:

| | | | | | Reference |
|---|---|---|---|---|---|
| | | | | | standard/Test |
| Test item | | Test Ex. | Comp. Ex. | Blank Ex. | method |
| Stock | Appearance | Homogeneous | Homogeneous | Homogeneous | Visual |
| solution | | and | and | and | inspection |
| | | transparent | transparent | transparent | |
| | Stability | Homogeneous | Homogeneous | Homogeneous | GB/T 6144 |
| | | and | and | and | |
| | | transparent | transparent | transparent | |
| Diluent | pH | 9.0 | 9.0 | 9.0 | GB/T 6144 |
| (20 wt %) | Transparency | Transparent | Transparent | Transparent | GB/T 6144 |
| | Corrosion | Grade A | Grade A | Grade A | GB/T 6144 |
| | Testing, LY12 | | | | Soaking at |
| | Duralumin | | | | 55° C. for 24 h |
| | Corrosion | Stainless, | Stainless, | Stainless, | JB/T 9189 |
| | test, cast iron | grade 0 | grade 0 | grade 0 | RT, 2 h |
| | filings | | | | |

The above results show that the product obtained by using the additive of the present application instead of triethanolamine and microbicide in the conventional formulation (comparative example) shows no difference from the conventional product in terms of apparent properties, stability, and corrosion resistance.

Test Result 2: stability testing (referencing to ASTM E2275 method, 30-day long cycle)

Namely, pH stability and bacterial corrosion resistance appearance test. The test example, the comparative example, and the blank metalworking fluid were diluted to 20 wt %, then added to a Francool® Emulsion Stability Tester for cyclic testing, to which 5 wt % of a plant odorant (containing naturally produced bacteria) was added. The relevant test results are shown below:

| | | Day 0 | Day 7 | Day 14 | Day 21 | Day 30 |
|---|---|---|---|---|---|---|
| Test | pH | 9.1 | 9.1 | 9.0 | 9.0 | 8.8 |
| Ex. | Nature | Opalescent/ | Opalescent/ | Opalescent/ | Very small | Small amount |
| | | odorless | odorless | odorless | amount of | of slightly |
| | | | | | slightly | yellowish oil |
| | | | | | yellowish oil | layer/odorless |
| | | | | | layer/odorless | |
| Comp. | pH | 9.1 | 9.0 | 8.9 | 8.9 | 8.3 |
| Ex. | Nature | Opalescent/ | Opalescent/ | Opalescent/ | Small amount | Small amount |
| | | odorless | odorless | odorless | of slightly | of slightly |
| | | | | | yellowish oil | yellowish oil |
| | | | | | layer/odorless | layer/odorless |
| Blank | pH | 9.1 | 8.5 | 7.8 | — | — |
| Ex. | Nature | Opalescent/ | Large | Blackening/ | — | — |
| | | odorless | amount of | odor | | |
| | | | floating oil | | | |
| | | | layer/odor | | | |

The above results show that over a 30-day cycle, the metalworking fluids with the additives of the present application (test example) had only a slight decrease in pH and only a small amount of unstable oil floats, which was similar to the pre-existing appearance of conventional metalworking fluids with the microbicide additive (comparative example). The pH of the comparative example did not decrease significantly in the first week, then decreased rapidly and dropped below 8.5 in 30 days, requiring timely replenishment of a new pH adjuster to continue use, while the samples of the test examples had better pH durability. In the blank example, no microbicidal component was added, which deteriorated rapidly after one week and the product failed completely. The test results show that the additive of the present application has good resistance in practical use, can better maintain the pH of the system, and can better help the system not be spoiled by bacteria to cause odor.

In summary, the phenolamine additive and the preparation method thereof provided by the present application have the following advantages.
1. The phenolamine additive has microbicidal properties, good stability, water solubility, and good stability when compounded into metalworking fluids, without causing any sedimentation or precipitation.
2. The phenolamine additives are all single-molecular species with good solubility, better antimicrobial effect, and stability.
3. With phenol as raw material, it can be directly dissolved in water and has high activity, and the reaction can be carried out quickly without using a strong alkali.
4. Methyl monoethanolamine is selected as the raw material, and methyl monoethanolamine is a pH regulator, so that the prepared phenolamine additive has a certain pH regulating ability and the stability can be further improved.
5. The preparation method of the present application is low in cost, simple in process, less in pollution, and easy to be popularized.

The above is only the embodiment of the application and does not limit the patent scope of the application. Any equivalent transformation made by using the description of the application, or directly or indirectly applied in relevant technical fields, is equally included in the patent protection scope of the application.

1. A phenolamine additive, comprising a compound of Formula (1), a compound of Formula (2), a compound of Formula (3), a compound of Formula (4), and a compound of Formula (5);

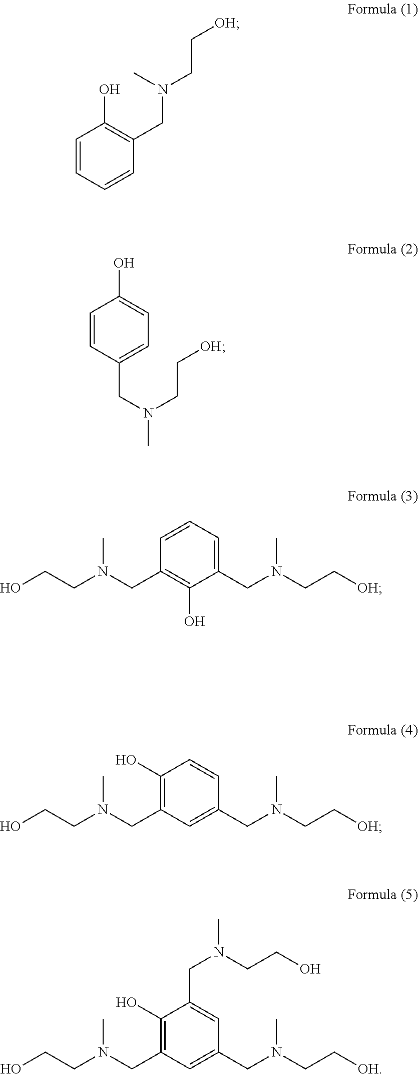 [SMILES: CN(CCO)Cc1ccccc1O ; CN(CCO)Cc1ccc(O)cc1 ; CN(CCO)Cc1cccc(CN(C)CCO)c1O ; CN(CCO)Cc1ccc(O)c(CN(C)CCO)c1 ; CN(CCO)Cc1cc(CN(C)CCO)c(O)c(CN(C)CCO)c1]

2. A preparation method of the phenolamine additive of claim 1, comprising the steps of reacting phenol, methyl monoethanolamine, and formaldehyde by a Mannich reaction to obtain the compound of Formula (1), the compound of Formula (2), the compound of Formula (3), the compound of Formula (4), and the compound of Formula (5).

3. The preparation method of the phenolamine additive of claim 2, comprising the steps of adding water, methyl monoethanolamine, phenol, and formaldehyde sequentially to a reaction vessel and reacting at 85-95° C. for 3.8-4.2 h to obtain the compound of Formula (1), the compound of Formula (2), the compound of Formula (3), the compound of Formula (4), and the compound of Formula (5).

4. The preparation method of the phenolamine additive of claim 3, wherein formaldehyde is added to the reaction vessel in batches.

5. The preparation method of the phenolamine additive of claim 2, wherein the mass ratio of the methyl monoethanolamine to phenol is 1 to 2.5:1.

6. The preparation method of the phenolamine additive of claim 2, wherein the mass ratio of the formaldehyde to phenol is 0.4 to 0.9:1.

7. Use of the phenolamine additive of claim 1 as a microbicide in a metalworking fluid.
